(19) United States
(12) Patent Application Publication
(10) Pub. No.: US 2006/0251541 A1
(43) Pub. Date: Nov. 9, 2006

(54) Scent delivery apparatus and method

(76) Inventor: Carmine Santandrea, Santa Barbara, CA (US)

Correspondence Address: MYERS BRADFORD, PLLC, 1731 WILLARD ST., STE. 502, WASHINGTON, DC, 20009, US

(21) Appl. No.: 11/482,140

(22) Filed: Jul. 7, 2006

Related U.S. Application Data
(63) Continuation-in-part of application No. 11/234,202, filed on Sep. 26, 2005.
(60) Provisional application No. 60/612,795, filed on Sep. 27, 2004.

(51) Int. Cl. A61L 9/04; A61L 9/12

(52) U.S. Cl. 422/5; 422/124; 705/10

(57) ABSTRACT

A method and scent device is described for enhancing multimedia advertising, sales or product promotion, public relations, entertainment or instructional systems by incorporating a scent dispersing apparatus mounted on the cage of a fan. The method includes steps of selecting a product or service, and delivering a multimedia advertising, sales promotion, public relations, entertainment, brand awareness or educational campaign which appeals to the sense of smell and one or more of the other senses. Methods are provided for measuring improvements in the effectiveness of multimedia advertising, sales or product promotion, public relations, entertainment or instructional systems occur when scent is incorporated into the campaign.

This application is a continuation-in-part of application Ser. No. 11/234,202, filed Sep. 26, 2005, which claims the benefit of the filing date of provisional application 60/612,795, filed on Sep. 27, 2004.

### FIELD OF THE INVENTION

This invention relates generally to an apparatus and methods for delivering a scent, especially in the context of multi-sensory advertising. Applications for the present invention include enhancing sales through multi-sensory based advertising, promotion, public relations, entertainment or instructional methods. More particularly, the invention relates to a scent cartridge mounted on fan for delivering one or more scents for the purpose of advertising, promotion, public relations, entertainment or instructional methods.

### BACKGROUND OF THE INVENTION

Advertising, sales promotional campaigns, product related public relations and product related instruction methods traditionally use film, television, radio, the Internet and print media as methods to persuade customers to purchase specific products. Television, movies, radio, the Internet and print advertising and promotional efforts are currently so ubiquitous that their effectiveness has been compromised even as the price of traditional advertising and promotion is rising [“Mass Media: Killing The Goose That Lays Golden Eggs”. BusinessWeek Online (Readers Report). Aug. 2, 2004]. In addition, film, TV, radio, Internet and print advertising primarily conventionally appeal to only two senses: sight and sound. As a result of rising advertising and sales promotion costs and declining effectiveness, there is a pressing need for new approaches to advertising, sales promotion, product public relations, story telling and instructional methods and there is a pressing need for methods to determine the effectiveness of advertising campaigns and communications.

The problem of effective communication advertising and promotional methods is particularly pressing in the commercial, retail environment where consumers are presented with a bewildering array of product choices and where advertisers have severely limited opportunities to convey a message. The Internet, one of the newest commercial environments, suffers from the same problems as the more traditional commercial arenas. On the Internet, customers are known to have a short attention span and limited patience, thereby limiting the ability of traditional advertising to convey product information and influence buying decisions.

One solution to the dilemma is to provide a multisensory, multimedia experience which enhances traditional visual and aural cues with at least olfactory cues. Scent, which acts on the limbic system of the consumer, evokes powerful emotional and memory experiences that can aid in persuading a consumer to purchase a product.

The power of scent to enhance or alter an experience is recognized in the art. U.S. Pat. No. 6,103,201, US20020130146A1 and U.S. Pat. No. 6,664,254 describe the use of scents in air fresheners to help cover offensive household odors. EP0495631A3, U.S. Pat. No. 5,220,741, U.S. Pat. No. 4,788,787 and WO0126818A1 describe the use of scents and pheromones as lures to attract animals to hunters or to repel insects. U.S. Pat. No. 5,963,302, U.S. Pat. No. 6,239,857, U.S. Pat. No. 6,152,829, US2001/0048641A1, US2004/0065748, U.S. Pat. No. 4,629,604 and U.S. Pat. No. 5,398,070 describe the application of scent to enhance movie, cinema, TV, video cassette and DVD viewing experiences, but these authors do not describe the use of entertainment media and scent for product-related instruction or public relations in the above applications.

The power of scent to alter mood or behavior for a person is also well recognized. EP0295129B1, WO9801175A1 and US2002/0124409A1 describe the medical and mood-altering characteristics of scent. U.S. Pat. No. 5,826,357 describes an artificial fireplace where fire scent sets the mood and enhances the experience. U.S. Pat. No. 6,669,092 describes a multimedia system to provide a virtual environment in order to reduce stress in a hospital or clinic. An advantageous application of scent delivery technology would be a personal scent delivery system which is capable of enhancing mood or behavior and reducing the stress level of a person within their home, office or similar enclosed space.

Multimedia advertising employing scent is known in the art. JP06328880A2, US20020158076A1, U.S. Pat. No. 6,557,778, U.S. Pat. No. 5,503,332 and U.S. Pat. No. 596,755 describe the use of scent to advertise and promote products in magazines and other print media, including transit tickets. US20030164268A1, US20030167284A1, US20030167464A1 and US20030177097A1 describe multimedia advertising applications for elevators where content is tailored to the expected interests of the elevator occupant. US20030129757A1 describes a food packaging system which allows a consumer to experience the scent characteristics of a finished product despite a sealed package. US20020047020A1 describes a vending system which allows a consumer to sample the sensory characteristics of a product before purchasing it. Similarly, ScentAir™ has reported dramatic increases in chocolate sales from vending machines when airborne chocolate scent is delivered into the region around the vending machine [Vending Times. 43(11) November, 2003].

One common example of a scent delivery device is an air freshener device in which a wick delivers a scent to an evaporative surface. WO03061716A1, U.S. Pat. No. 4,419,326, WO0230220A1, U.S. Pat. No. 5,857,620, U.S. Pat. No. 5,749,520, WO04014440A1 represent examples of the art. WO0230220A1 describes wick with specific pore characteristics for more effective scent delivery. U.S. Pat. No. 6,102,201 describes a scent bearing fan where scent is contained in the bearing of the fan. However, none of these devices describes a system in which the scent delivery system is incorporated into other mechanical components of the scent delivery system such as the fan itself.

Unique in-store multimedia scent delivery devices have also been described. U.S. Pat. No. 6,026,987 describes a mannequin which delivers scent and audio messages. U.S. Pat. No. 5,771,778 describes a jukebox which delivers music and scent. U.S. Pat. No. 3,844,057 describes lighted in-store display boxes which also incorporate scent. U.S. Pat. No. 5,069,876 describes a point-of-sale device incorporating audio, visual and scent cues. US20040103028A1 and US20030066073A1 describe in-store multimedia displays that sense the presence of a potential customer and change their displays in response to an approaching potential customer. US20040044564 describes a method in which in-store displays can be updated based on historical trends and current customer behavior using information supplied by a set of sensors located in the commercial environment.

Most known multimedia sensory experiences result from the delivery of a single scent. However, it is also widely understood consumers become insensitive to a scent after a short exposure. As a result, it would be advantageous to provide multimedia advertising, sales promotion, public relations, entertainment or instructional systems in which multiple scents are delivered in a scenting program where a scenting program comprises combinations of scents, scent delivery concentrations, scent delivery durations and scent delivery pauses. EP295120B1 describes a scent program for aroma therapy. U.S. Pat. No. 4,603,030 describes a scent program for a theater. US20040007787 describes a program for room scents.

Particular scent delivery devices often incorporate a scent and a dispersion method. For example, U.S. Pat. No. 6,834,847 describes the use of a fan and heater to disperse an air-borne scent. U.S. Pat. No. 6,511,531 describes an air freshening device comprising a filter element within the intake of the device housing, a motorized fan mounted within the device housing which draws contaminated air into the device, and forces air out of the device through an outlet having separate exhaust and scent discharge passages.

A number of scent delivery devices make various uses of fans as a vehicles for dispersing one or more scents. One class of fan-based scent delivery devices involves ceiling fans. U.S. Pat. No. 6,790,004, for example, describes a clip that can be attached to a blade of a ceiling fan. The clip contains an air freshener. US 2004247440 A1 is another example of a scent delivery device incorporating a scent receptacle attachable to one or more blades of a ceiling fan. This invention makes use of the movement of the scent receptacle while attached to the fan blade as the means for dispersing a scent located in a porous receptacle. Yet another example is U.S. Pat. No. 5,022,819, which describes scented “U” shaped clips that are securable to the lateral edges of ceiling fan blades by spaced parallel coil springs. Ceiling fans, however, are not as readily accessible as other fans, for example floor mounted fans, in the typical sales environment. Also, ceiling fans typically direct air flow in a single static direction.

In addition to ceiling fan scent devices, another class of fan-based scent delivery systems incorporates a fan and ductwork directing a forced air stream across a scent medium. These tend to me more complicated devices, frequently incorporating many parts and requiring complicated manufacture. For example, U.S. Pat. No. 6,834,847 by Bartsch describes a scent dispensing device using a fan and a heater to disperse a scent. As typical with many of these an similar devices, they are fairly complicated and not easily adapted to the multi-sensory sales environment, where ease of use, replacement of scent, and simple controls are important factors.

Yet another class of scent dispersing devices incorporates the scent into the fan itself. This is perhaps the most useful class of scent devices for application in multi-sensory advertising developed to date. US application 20050275118 to Chen is one example where the scent is located in the hub of the fan rotor connected to the blades. This approach offers a simple an elegant way to introduce scent into a sales environment. However, it may not provide the best option where scents need to be frequently changed or replaced. Also, integration of the scent into the inner mechanism of the fan may result in manufacturing and distribution complications. Another solution has been recognized in the use of a scent pad or other self-contained scent dispenser mounted on part of a fan. U.S. Pat. No. 6,109,874 to Steiner is one example where a scent pad is mounted on a support for the fan. It is recognized that the scent source should be positioned so that rotating fan blades will disseminate the scent. Thus, inventions such as this tend to place the scent source in a location near the blades, or even on the blades. Mounting scent directly on the blades may interfere with the balance of the blades and blade rotation. Locations farther from the blades do not offer as good dispersion rates. There has been a long-standing need to find an optimal location for the scent source offering maximum dispersion and that is structurally sound and easily accessible. The specific use of a fan grill or cage previously may not have seemed like a possibility due to problems mounting a scent source on this type of structure. One problem is that the fan cage does not offer a regular surface for mounting a structure such as a scent pad. Another problem is that air flow through the cage applies a force opposing placement of a scent source such as a pad. The center of the cage frequently has a flat section with minimal opposing force, but also lacks air flow. Previously it may not have been thought possible to mount a scent source at this location due to insufficient air circulation across the scent source. As set forth in further detail below, the present invention has found a previously unknown way to accomplish these various goals required for scent dispensing using a method likely considered counterintuitive.

Even with an effective scent delivery system, it is necessary to conduct careful research to determine that the introduction of a particular scent or scents has a desired effect.

It is widely believed that a cue mismatch where an inappropriate scent is delivered in a multimedia presentation results in consumer dissatisfaction. As a result, it is desirable to provide methods to assure that the elements of a multi-sensory, multimedia campaign be linked, preventing incorrect associations from developing in a consumer's mind. U.S. Pat. No. 6,669,092 describes a method to coordinate elements of a virtual environment employing a light box, audio player and a scent distributor by electronically tagging each element.

Rising advertising costs and decreasing advertising effectiveness argue for choosing to use only cost-effective advertising. However, none of the examples provide means to objectively measure the success or failure of the advertising, sales promotion or public relations or instructional campaign which employs scent. As a result, it would be advantageous to provide multimedia advertising, sales promotion, public relations, entertainment or instructional programs which measure the effectiveness of the program.

### OBJECT OF THE INVENTION

It is therefore an object of the present invention to provide a method for enhancing the sale of a product by providing multimedia stimulus methods employing one or more scents in conjunction with media that evoke responses from one or more of the other senses.

Another object of the invention is to provide a measurement of the effectiveness of an advertising, sales promotion, public relations, entertainment or instructional method.

Another object of the invention is to provide brand enhancement by combining scent with multimedia experience.

Still another object of the invention is to provide an enhanced Internet shopping experience by combining scent with Internet based advertising, sales promotion, public relations, entertainment or instructional methods.

Still another object of the invention is to provide a method to coordinate multimedia advertising, sales promotion, public relations, entertainment or instructional materials.

Yet another object of the invention is to provide a distinctive personal environment by local delivery of scent in a scent program.

### SUMMARY OF THE INVENTION

The present invention provides a system for multi-sensory, multimedia advertising, sales promotion, education or product public relations campaign and a method to rapidly assess the effectiveness of an advertising campaign. The method provides both scent cues and more traditional non-scent based cues to help influence a customer toward the purchase of a selected, target product.

The present invention contains several elements that in combination provide a uniquely accountable advertising method. In general, the method comprises a product which is displayed and offered for sale in a commercial environment or zone, one or more scents corresponding to the product which are delivered into a scenting zone according to a scent program, non-scent advertising or sales promotional materials, educational materials or product public relations materials and strategically located counters which measure the effectiveness of the advertising or sales promotion campaign.

The present invention may be applied to traditional retail commercial environments such as grocery stores, gas stations, pharmacies, department stores and the like. Alternatively, the method may be equally effectively applied to sales of products on the Internet where a potential customer is physically separated from a direct product experience.

The present invention may be used to advertise or promote any product, but the method is especially well suited to products where the normal scent is masked by packaging. Examples of food products to which the method is well suited include coffee, rolls, chocolate bars, frozen foods and the like. Other examples of suitable products include laundry products, new or used cars, lawn products such as fertilizers, cosmetics and the like. It will be understood by those skilled in the art that many other examples are also possible.

The present invention may also be applied to brand building activities in which a customer is exposed to a product in an environment containing one or more scents corresponding to the brand and other non-scent based advertising, sales promotion, educational or product public relations material. A result of a brand building activity is to induce a potential customer to associate a specific product brand with a scent or scent program.

The present invention may also be applied to development of a personal brand or personal aura in which a unique scent combination is delivered into a scenting region, inducing a response. The responses induced include relaxation, the presence of specific person in the room and other possible responses.

### BRIEF DESCRIPTION OF THE DRAWINGS

FIG. 1 is a flow-chart showing an advertising or promotional method with an effectiveness measurement method comprising a product in a commercial zone, advertising or other promotional materials located in the commercial zone, a detector in the commercial zone which identifies a potential customer entering the zone, a detector at the point of purchase which counts sales of the product, a time measurement device, and a comparator through which the efficiency of an advertising program can be measured.

FIG. 2 is a flow-chart showing a multimedia advertising or promotional method with an effectiveness measurement method comprising a product in a commercial zone, a detector in the scenting zone which identifies a potential customer entering the scenting zone, a scent delivery system which delivers one or more scents in a scenting program related to the non-scent advertising materials located in the commercial zone, a detector at the point of purchase which counts sales the product, a time measurement device, and a comparator through which the efficiency of an advertising program can be measured.

FIG. 3 is a flow chart showing a multimedia advertising or promotional method with an effectiveness measurement method comprising an advertising zone which contains non-scent related advertising or promotional materials for a product, a scenting zone into which one or more scents related to a product are delivered according to a scenting program, a product zone, which is optionally remote from the scenting and advertising zones, a first counter operationally connected to a scent dispersal system and a customer detector, an automatic door opener which is operationally connected to the scent dispersal system and a customer detector, a detector at the point of purchase which counts sales of the product and a comparator through which determines the efficiency of an advertising program can be measured.

FIG. 4 is an exploded view of a strip-like scent delivery device. The scent delivery element is configured as a strip, sheet or similar shape with substantial surface area. The strip-like scent delivery device package comprises a polymeric material configured with an extended surface area, a scent or scents which are characteristic of a selected product or brand which are compatible with the polymeric material and which can be incorporated into the polymeric material. The polymeric material is chosen to release scent at a controlled rate. A removable covering device or layer which is impermeable to scent sandwiches the scent delivery element and permits scent delivery only upon removal or breakage. Optionally, one or more exterior surface of the device can be coated with a suitable adhesive, allowing the completed device to stick to a package or product surface. When a shipping package is readied for display, the barrier seal is broken and scent is released. The scent material is attached not so much to the individual packages but the shipping case or display or to a price strip located near the product display.

FIG. 5 is an elevation view of a scent delivery device which fits into a standard light fixture. The device can be turned on or off using a standard light switch or sensor such as a motion detector. The fan blade of the scent delivery device is preloaded with a scent or scents. Or a scent cartridge can be in the proximity of the fan blade where the movement of the fan blade draws the scent from the cartridge. This can also be in the form of a scented grill in front of the fan. When the light switch is turned on, the motor is energized and the fan blades turn. Air flow over the fan blades delivers the selected scent into a selected area. Optionally, the motor can generate heat facilitating vaporization of scent. Also optionally, a separate heater to facilitate vaporization of scent can be incorporated into the device. This could be a light or projection lamp incorporated in the device.

FIG. 6 is a perspective view of fan having a protective cage intended to be used with a scent delivery apparatus according to the present invention.

FIG. 7a is a perspective view of a scent delivery device made by attaching a scent receptacle to the protective cage of a fan.

FIG. 7b is an exploded view of a scent receptacle designed to be placed on a protective grill of a fan.

FIG. 8a is a perspective view of a scent receptacle cover.

FIG. 8b is a top view of a scent receptacle cover.

FIG. 8c is a perspective view from the side of a scent receptacle cover.

FIG. 9a is a perspective view of a scent cartridge designed to be placed within the scent receptacle of the present invention.

FIG. 9b is a side perspective view of a scent cartridge designed to be placed within the scent receptacle of the present invention.

FIG. 10a is a perspective view of a scent receptacle base.

FIG. 10b is a perspective view from the side of a scent receptacle base.

FIG. 10c is a bottom view of a scent receptacle base.

### DETAILED DESCRIPTION OF THE INVENTION

Definitions

The following terms have meaning described below and are used throughout this specification:

Advertising Zone. An advertising zone is an area containing advertising materials, product promotional materials, product educational materials, brand awareness activities or materials, or public relations materials for a product, company or group of people. Advertising is intended to be construed in its broadest terms to include materials and processes related to advertising, product promotion, product education, and product public relations materials. Non-limiting examples of advertising materials include signs, leaflets, gobos, light boxes, holographic displays, audio tapes or recordings, computer displays and the like.

“Non-scent” used in the context of multi-media advertising means a combination of advertising materials which stimulate primarily one or more of the senses of taste, touch, hearing or sight, but which are not primarily directed toward the sense of smell.

Commercial Zone. A commercial zone is an area in which a product is displayed or presented, and offered for sale. Typically, a commercial zone will be found in a retail store or other establishment. Non-limiting examples of establishments which contain commercial zones include a gas station, a bakery, a grocery store, a drug store, a cosmetics retailer. Vending machine locations contain commercial zones. In cases where a product is offered for sale, a commercial zone can also include areas in a theater, a concert, a movie house, a cinema, or a lecture hall. Commercial zones may be confined to a phone or fax as well. Similarly, a commercial zone can include a home when a product is offered for sale using any suitable media.

Sales Environment. A sales environment is any location in which sale of a product occurs. Typically a sales environment contains a commercial zone where a product is displayed and offered for sale, but a sales environment also contains other facilities such as entry ways, customer service areas, product check-out areas and the like. A sales environment may contain a scenting zone or an advertising zone.

Scent. Scent is broadly defined to include aromas, fragrances, perfumes and pheromones. Scent can also be a molecule that removes mal odor molecules of smell. A scent is typically a volatile or at least nominally vaporizable material that stimulates receptors located primarily in the nose. Scents may be powerful stimulants. A scent may affect the limbic system and evoke significant physiological responses, emotional reactions and memories that can aid advertisers in delivering an advertising message if the scent is properly chosen and properly delivered. A substantial variety of scents are available from commercial vendors such as Lebermuth (South Bend, Ind.), Aroma Tech (Somerville, N.J.), Orpheus, Ltd. (Rockway, N.J.) or ScentAir (Santa Barbara, Calif.). These products are typically used in candles, aroma therapy, advertising, and air-modifications such as air fresheners and the like.

Pheromones. A pheromone is a chemical secreted externally by animals or insects that influences the physiology or behavior of other animals of the same species. Human pheromones may significantly affect human behavior if properly presented, and represent an essentially untapped field for advertisers.

Scenting Zone. A scenting zone is an area into which one or more scents are delivered. The size of a scenting zone can be adjusted depending on the product being advertised and the establishment in which the advertising is occurring.

Scenting Program. A scent program comprises a combination of scents, scent delivery concentrations, scent delivery durations and scent delivery pauses. A scent program can be considered analogous to a musical score where scent notes are similar to music notes. The scent delivery duration may be similar to playing whole, half or quarter notes, etc., and scent delivery pauses may be equivalent to musical rests.

Scent Delivery Device. Delivery devices for scents have one or more key elements: a volatile scent or scent solution; a heating device; a fan or other scent transport device, and appropriate ducts, dampers or tubes or other mechanical means to deliver the scent to a desired location. Examples of scent delivery devices include Green, D. in U.S. Pat. No. 6,103,201, which describes a fan with various shapes, Manne, J. in US20020018181A1 describes a system of tubes and a mask for delivering scent directly to a person's nose. Wohrle, G, in US20030107139A1 a plurality of cartridges containing scented fluids, scents in pockets in a tray and a heating systems to evaporate or vaporize a scent or scents. Shaahin, S. in WO03068300A1 describes a scent delivery system containing temperature control to avoid formation and inhalation of harmful degradation products. Prueter; D. in U.S. Pat. No. 6,631,888 describes a battery operated fragrance delivery device containing a fan where the cap contains ductwork and an on-off switch. Stenico, F. in WO03086487A1 which describes a delivery system with a wick, an evaporator, a fan and a system of louvers. In the present invention, any suitable combination of scent delivery elements may be employed.

Time-slice. A time-slice is a time period during which the efficiency of an advertising campaign is assessed. A time-slice duration is chosen for convenience of the measurement. Typically, a time-slice can be measured in seconds, minutes, hours, days, weeks, months or years.

Advertising Method with an Effectiveness Measurement

With advertising costs rising and advertising effectiveness diminishing, a method for advertising which provides a measurement of the effectiveness of an advertising campaign is needed. The effectiveness measurement provides managers with objective tools to decide whether to continue an advertising or promotional campaign or communications message.

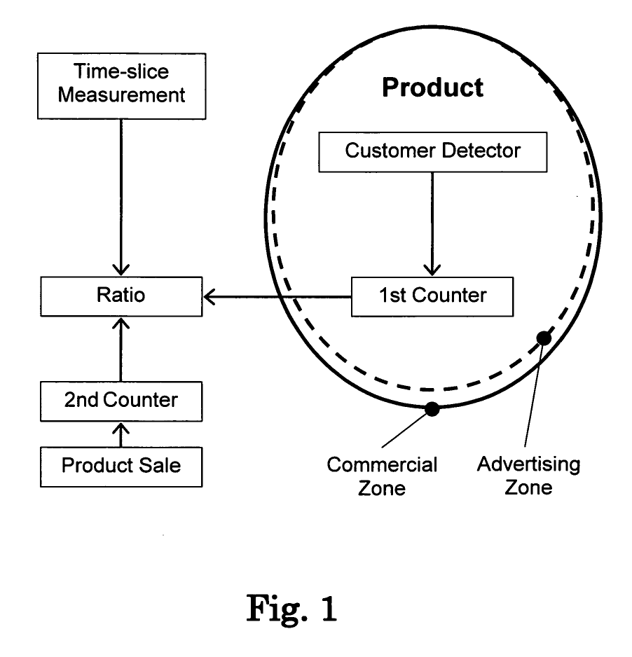

FIG. 1 shows an advertising method with a measurement of effectiveness. The method comprises selecting a product for the campaign and a duration or time-slice for the campaign. The product is displayed for sale in a commercial zone within a sales environment. A region containing the product, and an advertising zone within the commercial zone, contain a detector to determine when a potential consumer enters the advertising zone, thereby triggering an increment of the first counter. A second counter is connected to or acquired from equipment at the point-of-purchase and is triggered when the selected product is purchased. The first and second counters are connected to a comparator from which a measurement of effectiveness is derived. A comparator can be incorporated into one of the counters.

A time-slice is chosen for convenience of the measurement and is selected to be long enough to accurately assess the effectiveness of the advertising or sales promotion campaign. The duration of a time-slice is preferably measured in seconds, minutes or years. More preferably, a time-slice is measured in hours, days, weeks or months.

In order to measure the effectiveness of an advertising or sales promotion campaign, it is necessary to create an objective measurement of consumer interest in the product. One measurement is to define a scenting, advertising or commercial zone around a product and determine how frequently a consumer touches, views, considers or is physically near a product for sale vs. how often the same product is sold. While it is possible to station a person with a counter near a product display, the solution is typically cost prohibitive and prone to human errors. A more cost-effective and more accurate solution is to provide a sensor which determines when a potential customer enters the commercial zone.

A variety of sensors are capable of sensing the presence of a potential customer in a commercial, advertising or scenting zone. The general classes of sensors that are capable of detecting a potential customer in a commercial zone include heat or infrared sensors, microwave sensors, capacitance sensors, electromagnetic field sensors, motion sensors, light sensors, retinal or iris scan sensors, biometric sensors, pressure sensors, audio or ultrasonic sensors, and digital images.

The method of measuring the effectiveness of an advertising campaign are related to the scientific method in which results for a control group are compared to the results for an experimental group. To initiate an effectiveness measurement, a control, baseline or first sales rate is measured before a new advertising, sales promotion, educational or public relations campaign is initiated. Then, the campaign is initiated and a second sales rate is determined. The sales increment between the second and first sales rate is a quantitative measurement of the effect of the campaign.

FIG. 1 illustrates an advertising, sales promotion, educational or public relations campaign effectiveness measurement process. A selected product is displayed for sale in an advertising zone within a commercial zone of a sales environment. To measure the effectiveness of a campaign, a first sales rate is measured, as exemplified in the following steps:
1. Both the first and second counters are set to zero at the beginning of a first measurement period, a time-slice (TS1).
2. When a potential customer enters the advertising zone, the customer detector identifies appropriate behavior. Appropriate behavior is predefined and includes, but is not limited to, changes in eye, hand or body movements, changes in heart rate, changes in the electrical or electromagnetic spectrum of the potential customer or changes in the physical position of a potential customer.
3. A first counter is triggered and the counter counts all potential customers according to appropriate preset criteria with in the time-slice, forming a value FCV1.
4. A second counter is located at a check-out or other location where a product is purchased. The second counter is triggered when the selected product is purchased and counts accumulate during the time-slice forming a value, SCV1.
5. If time-slice for the first sale rate, baseline measurement and the second sale rate measurements are constant, the first sales rate, FSR, can be calculated as follows:
   FSR1=SCV1/FCV1 Eq. 1
6. If the time-slices for the first sales rate measurement and second sales rate measurement are not equal, the first sale rate measurement can be calculated as
   FSR2=SCV1/FCV1/TS1 Eq. 2
7. When baseline measurements are completed, the advertising campaign is installed in the advertising zone. As the campaign is initiated both counters are set to zero in order to initiate the second sales rate measurement, initiating a second time-slice measurement, TS2.
8. When a potential customer enters the advertising zone containing the advertising campaign, the customer detector identifies appropriate behavior. Appropriate behavior is predefined but is the same behavior measured in Step 2.
9. A first counter is triggered and the counter counts all potential customers according to appropriate preset criteria with in the time-slice, forming a value FCV2.
10. A second counter is located at a check-out or other location where a product is purchased. The second counter is triggered when the selected product is purchased and counts accumulate during the time-slice forming a value, SCV2.
11. If time-slice for the first sale rate, baseline measurement and the second sale rate measurements are constant, the second sales rate, SSR, can be calculated as follows:
   SSR1=SCV2/FCV2 Eq. 3
12. If the time-slices for the first sales rate measurement and second sales rate (ssr) measurement are not equal, the second sale rate measurement can be calculated as follows:
   SSR2=SCV2/FCV2/TS2 Eq. 4
13. The increment in sales (ISR) due to the advertising method employed can be estimated by comparing the first sales rate with the second sales rate. If the time-slices, TS1 and TS2, are the same, the following equation defines the sales increment.
   ISR1=SSR1−FSR1 Eq. 5.
14. If the time-slices, TS1 and TS2, are unequal, the following equation defines the sales increment.
   ISR2=SSR2−FSR2 Eq. 6.
   ISR2 multiplied by a time-duration is an estimate of the sales increment due to adding scent to the multimedia advertising campaign, and can be directly monetized for a merchant or other advertising customer.

Several variations of the above themes are possible. Steps 1-6 and Steps 7-14 can be applied sequentially in a single sales environment. Optionally, Steps 1-6 and Steps 7-14 can be conducted in separate sales environments if they have been shown to be sufficiently equivalent. Optionally, a cross-check design can be employed where Steps 1-6 alternate with Steps 7-14 several times to provide repeated measurements and increased accuracy. Optionally, a cross-over design can increase measurement accuracy; Steps 7-14 can be employed a location 1 and Steps 1-6 conducted at location 2, and then Steps 7-14 go to location 2 and Steps 1-6 go to location 1. Many other procedural variations and test designs are possible and will be understood by those skilled in the art as being within the scope of the invention.

In the process described, the counters and timers can be located wherever it is convenient. Both counters and timers can be located at a remote computer and connected to the sales environment by any convenient method including, but not limited to, hard wires, telephone links, infrared links and radio or microwave links. In addition, the calculations described above can be performed manually after reading the counters and timers or they can be performed on a computer using a variety of programs.

A Multimedia Sales Enhancing Campaign with Scent Delivery

Advertising is now so ubiquitous that the effectiveness of many advertising methods is diminishing. One solution to the problem is to incorporate scent into a multimedia adverting, sales promotion, educational or public relations campaign. Scent uniquely triggers memories and can help differentiate one product from another.

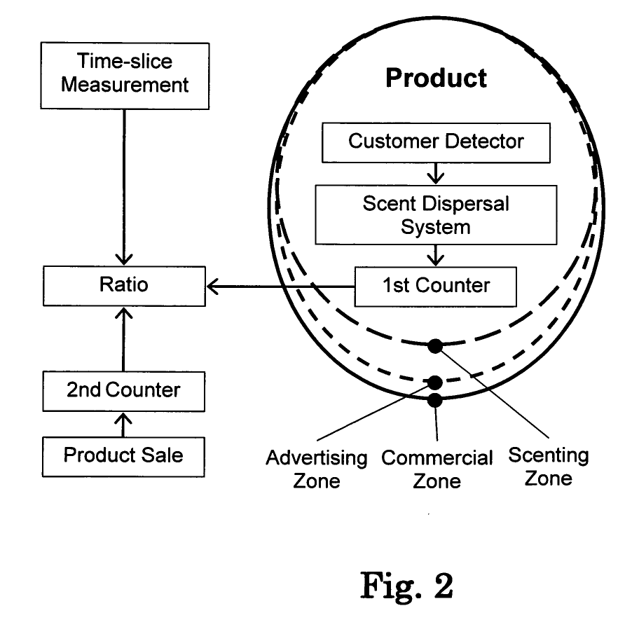

A method for a multimedia advertising system employing scent is diagrammed in FIG. 2. Initially, a product is selected and a scent or scents related to the product are selected, compounded or formulated. The selected product is located in a sales environment and is displayed and offered for sale in a commercial zone which also contains an advertising zone and a scenting zone.

Typically, a scenting zone is located near the product and an advertising zone is also located near the product. The scenting zone and advertising zone can overlap, but need not be coincident.

In this embodiment, a scent dispersal system is located to deliver a scent or scent program into a scenting zone. The scent dispersal system can be chosen from among several commercial scent delivery systems such as those supplied by ScentAir™. Alternatively, a specialized scent delivery system can be constructed to evaporate or vaporize a scent and deliver scent into a scenting zone. In a most preferred embodiment, the scent delivery device is capable of delivering multiple scents for pre-selected, variable times and to pre-selected concentrations to a scenting zone, a specific contained zone within a sales environment.

Optionally, the scent delivered by the scent delivery device is a single scent or multiple scents. With multiple scents, a scent delivery program is possible where delivery involves multiple scents for pre-selected, variable times and to pre-selected concentrations to a scenting zone, a specific, contained zone within a sales environment. A scent program may be considered analogous to a musical score where a scent note is the same as a music note; the scent delivery duration is similar to playing whole, half or quarter notes, and scent delivery pauses are equivalent to musical rest.

Scent delivery or a scenting program is trigged when a customer is detected in the scenting zone. In one embodiment of the invention, scent delivery can be triggered by a person who is stationed to observe the scenting zone. However, in a more preferred embodiment of the invention, an automatic sensing device employed to detect the presence of a potential customer in the scenting zone. A variety of automated methods can be used to detect a customer in a scenting zone including, but not limited to, heat or infrared sensors, microwave sensors, capacitance sensors, electromagnetic field sensor, motion sensors, light sensors, retinal or iris scan sensors, biometric sensors, pressure sensors, audio or ultrasonic sensors or digital visuals.

Optionally, the customer can be asked to provide permission before scent is delivered. Permission to deliver a scent or scent program can be provided by a variety of methods including, but not limited to, eye movement, hand movement, other body movements, by pushing a button, by pulling a lever, by audio cues, by electrical or electromagnetic changes near or in the body, by standing in a specified location, by passing through a specific location, by passing by a specific location, by body heat, by pupil dilation, body language or by body temperature changes or by heart rate changes.

A Method for Measuring the Effectiveness of Multimedia Sales Enhancing Campaign with Scent Delivery

Since advertising in general is becoming ever less effective, there is a need to assess and demonstrate the effectiveness of scent in delivering a sales boost. The effectiveness of a multimedia sales enhancing campaign that is augmented with scent delivery can be measured using a process that is similar to a scientific experimentation process where the sales of a control group in which a multimedia advertising system without scent is compared to the sales of an experimental group in which a multimedia advertising system is enhanced with scent.

The steps of the measurement are similar to those described for an advertising method with an effectiveness measurement and is diagrammed in FIG. 2. Initially, a product is located in a sales environment which contains an advertising zone, which contains non-scent sales enhancing material, and a scenting zone where the scenting zone also contains a scent delivery device that is capable of delivering a single scent or a scent program that is related to the product and a detector that identifies a customer within the scenting zone.

For the example that follows, a first sales rate is measured with the non-scent related multi-media sales enhancing campaign in place and active, but the scent delivery device turned off. The second sales rate is equivalent but the scent delivery device is turned on.
1. Both the first and second counters are set to zero at the beginning of a first measurement time-slice (TS1).
2. When a potential customer enters the scenting zone, the customer detector identifies appropriate behavior. Appropriate behavior is predefined and includes, but is not limited to, changes in eye, hand or body movements, changes in heart rate, changes in the electrical or electromagnetic spectrum of the potential customer or changes in the physical position of a potential customer.
3. A first counter is triggered and the counter counts all potential customers according to preset criteria with in the time-slice, forming a value FCV1.
4. A second counter is located at a check-out or other location where a product is purchased. The second counter is triggered when the selected product is purchased and counts accumulate during the time-slice forming a value, SCV1.
5. If time-slice for the first sale rate, baseline measurement and the second sale rate measurements are constant, the first sales rate, FSR, can be calculated as follows:
   FSR1=SCV1/FCV1 Eq. 1
   6. If the time-slices for the first sales rate measurement and second sales rate measurement are not equal, the first sale rate measurement can be calculated as
   FSR2=SCV1/FCV1/TS1 Eq. 2
7. When baseline measurements are completed, the advertising campaign is augmented with scent by turning on the scent delivery device. As the next phase of the sales enhancing campaign is initiated, both counters are set to zero in order to initiate the second sales rate measurement which also initiates a second time-slice measurement, TS2.
8. When a potential customer enters the scenting zone containing the advertising campaign, the customer detector identifies appropriate behavior and a scent or scent program is delivered. Appropriate behavior is predefined but is the same behavior measured in Step 2.
9. A first counter is triggered and the counter counts all potential customers according to appropriate preset criteria with in the time-slice, forming a value FCV2.
10. A second counter is located at a check-out or other location where a product is purchased. The second counter is triggered when the selected product is purchased and counts accumulate during the time-slice forming a value, SCV2.
11. If time-slice for the first sale rate, baseline measurement and the second sale rate measurements are constant, the second sales rate, SSR, can be calculated as follows:
   SSR1=SCV2/FCV2 Eq. 3
12. If the time-slices for the first sales rate measurement and second sales rate (SSR) measurement are not equal, the second sale rate measurement can be calculated as follows:
   SSR2=SCV2/FCV2/TS2 Eq. 4
13. The increment in sales (ISR) due to scent augmentation of the multimedia advertising method can be estimated by comparing the first sales rate with the second sales rate. If the time-slices, TS1 and TS2, are the same, the following equation defines the sales increment.
   ISR1=SSR1−FSR1 Eq. 5.
14. If the time-slices, TS1 and TS2, are unequal, the following equation defines the sales increment.
   ISR2=SSR2−FSR2 Eq. 6.
   ISR2 multiplied by a time duration is an estimate of the sales increment due to adding scent to the multimedia advertising campaign, and can be directly monetized for a merchant or other advertising customer.

Several variations of the theme are possible. Steps 1-6 and Steps 7-14 can be applied sequentially in a single sales environment. Optionally, Steps 1-6 and Steps 7-14 can be conducted in separate sales environments if they have been shown to be sufficiently equivalent. Optionally, a cross-check design can be employed where Steps 1-6 alternate with Steps 7-14 several times to provide repeated measurements and increased accuracy. Optionally, in a cross-over design can increase measurement accuracy, Steps 7-14 can be employed a location 1 and Steps 1-6 conducted at location 2, and then Steps 7-14 go to location 2 and Steps 1-6 go to location 1. Many other procedural variations and test designs are possible and will be understood by those skilled in the art as being within the scope of the invention.

In the process described, the counters and timers can be located wherever it is convenient. Both counters and timers can be located on a remote computer and connected to the sales environment by any convenient method including, but not limited to, hard wires, telephone links, infrared links and radio or microwave links. In addition, the calculations described above can be performed manually by after reading the counters and timers or they can be performed on a computer using a variety of programs.

A Multimedia Method Employing Scent for Enhancing the Sale of a Product Near a Door of a Sales Environment

In advertising, it is sometimes desirable to influence a consumer to purchase a product using a multimedia advertising method as he or she enters a store or other commercial establishment. Scent, as a powerful memory jogger, can aid in planting a desire to purchase a specific product, but scent is even more effective when combined with a multimedia advertising message.

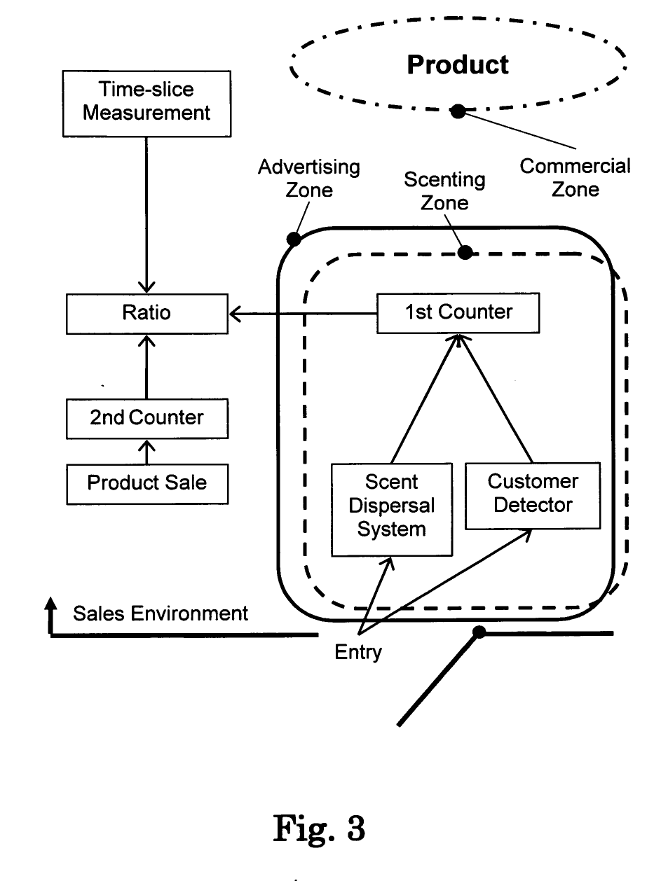

FIG. 3 is diagram of a multimedia sales enhancing method employing scent for advertising a product near an entrance of a sales environment. In this embodiment of a multimedia advertising method employing scent, a product is selected and is displayed for sale in a commercial zone of a sales environment. Optionally, the commercial zone can be located near the door of the sales environment if it is desirable. One embodiment of the present invention incorporates the use of a door-opening mechanism which may be operably connected to the scent dispersal system near an entrance. By “operably connected,” one will understand that a controller, program, or mechanical device allows for activation of the scent dispersal system after activation of the door opening mechanism. Optionally, the commercial zone is remote from the door of the sales environment, but if the commercial zone is remote from the entrance, the advertising zone contains instructions to find the product in the sales environment. Examples of instructions include, but are not limited to, people, signs or light boxes, handouts, audio sound, TV and the like.

An advertising zone, which contains non-scent, sales-enhancing materials, is located near the entrance to the sales environment. Additionally, located near the entrance of a sales environment is a scenting zone. The scenting zone also contains a scent delivery device that is capable of delivering a single scent or a scent program that is related to the product and a detector that identifies a customer within the scenting zone.

In this embodiment, a scent delivery system is located to deliver a scent or scent program into a scenting zone. The scent dispersal system can be chosen from among several commercial scent delivery systems such as those supplied by ScentAir™. Alternatively, a specialized scent delivery system can be constructed to evaporate or vaporize a scent and deliver scent into a scenting zone. In a most preferred embodiment, the scent delivery device is capable of delivering multiple scents for pre-selected, variable times and to pre-selected concentrations to a scenting zone, a specific, contained zone within a sales environment.

Optionally, the scent delivered by the scent delivery device is a single scent or multiple scents. With multiple scents, a scent delivery program is possible where delivery involves multiple scents for pre-selected, variable times and to pre-selected concentrations to a scenting zone, a specific, contained zone within a sales environment.

A door opener may be connected to the scent delivery system or a sensor at an entrance. Scent delivery or a scenting program is trigged when a customer opens or triggers the sensor at an entrance in the sales environment. Optionally, scent delivery is triggered when the customer opens or triggers the opening of a door in the sales environment and is detected in the scenting zone, thereby avoiding scent waste by scenting an unoccupied scenting zone or annoying people who are nearby but are not customers.

In one embodiment of the invention, scent delivery can be triggered by a person who is stationed to observe the scenting zone. However, in a more preferred embodiment of the invention, an automatic sensing device employed to detect the present of a potential customer in the scenting zone. A variety of automated methods can be used to detect a customer in a scenting zone including, but not limited to, heat or infrared sensors, microwave sensors, capacitance sensors, electromagnetic field sensor, motion sensors, light sensors, retinal or iris scan sensors, biometric sensors, pressure sensors, audio and ultrasonic sensors.

A Method for Measuring the Effectiveness of a Multimedia Sales Enhancing Campaign Employing Scent for a Product Near an Entrance of a Sales Environment

The growing ineffectiveness of traditional advertising media is forcing advertisers to consider new approaches. Scent is a unique and powerful memory jogger which can be combined with other advertising and promotion media to influence a potential customer. Sometimes it is especially useful to influence a potential customer as he or she enters a sales environment. However, due to growing cost constraints advertisers will need to prove the effectiveness of their advertising and promotion campaigns to retain stores and mangers in the future.

Measuring the effectiveness of a multimedia sales enhancing campaign employing scent for a product located near an entrance of a sales environment is diagrammed in FIG. 3. The advertising, commercial and scenting zones are arranged as described above. A scent delivery system is directly or indirectly connected to a door opener and customer detector as described above. To enable measurement of the effectiveness of scent in a multimedia advertising campaign, time measurement devices are arranged and counters are arranged in the scenting zone and in the sales environment where product is purchased.

In order to conduct a measurement of the effectiveness of the multimedia advertising campaign employing scent near the entrance of a sales environment, the following steps may be taken:
1. To take a baseline measurement, both the first and second counters are set to zero at the beginning of a first measurement time-slice (TS1).
2. When a potential customer enters the sales environment, a multimedia advertising campaign enhanced by scent delivery is in place, but the scent delivery device is disabled in order to develop a baseline first sales rate.
3. When a customer enters sales environment and is detected in the scenting zone, a first counter is triggered and the counter counts all customers who enter the sales environment within the time-slice, forming a value FCV1.
4. A second counter is located at a check-out or other location where a product is purchased. The second counter is triggered when the selected product is purchased and counts accumulate during the time-slice forming a value, SCV1.
5. If time-slice for the first sale rate, baseline measurement and the second sale rate measurements are substantially equal, the first sales rate, FSR, can be calculated as follows:
   FSR1=SCV1/FCV1 Eq. 1
6. If the time-slices for the first sales rate measurement and second sales rate measurement are not substantially equal, the first sale rate measurement can be calculated as follows:
   FSR2=SCV1/FCV1/TS1 Eq. 2
7. When baseline measurements are completed, the advertising campaign is augmented with scent by turning on the scent delivery device. The next phase of the sales enhancing campaign is initiated and both counters are set to zero in order to initiate the second sales rate measurement which also initiates a second time-slice measurement, TS2.
8. When a potential customer enters of the sales environment and optionally is also detected in the scenting zone as well a scent or scent program is delivered, a first counter is triggered and the counter counts all potential customers according to appropriate preset criteria within the time-slice, forming a value FCV2.
9. A second counter is located at a check-out or other location where a product is purchased. The second counter is triggered when the selected product is purchased and counts accumulate during the time-slice forming a value, SCV2.
10. If time-slice for the first sale rate, baseline measurement and the second sale rate measurements are constant, the second sales rate, SSR, can be calculated as follows:
   SSR1=SCV2/FCV2 Eq. 3
11. If the time-slices for the first sales rate measurement and second sales rate (SSR) measurement are not equal, the second sale rate measurement can be calculated as follows:
   SSR2=SCV2/FCV2/TS2 Eq. 4
12. The increment in sales (ISR) due to scent augmentation of the multimedia advertising method can be estimated by comparing the first sales rate with the second sales rate. If the time-slices, TS1 and TS2, are the same, the following equation defines the sales increment.
   ISR1=SSR1−FSR1 Eq. 5.
13. If the time-slices, TS1 and TS2, are unequal, the following equation defines the sales increment.
   ISR2=SSR2−FSR2 Eq. 6.
   ISR2 multiplied by a time-duration is an estimate of the sales increment due to adding scent to the multimedia advertising campaign, and can be directly monetized for a merchant or other advertising customer.

Several variations on the theme are possible. Steps 1-6 and Steps 7-14 can be applied sequentially in a single sales environment. Optionally, Steps 1-6 and Steps 7-14 can be conducted in separate sales environments if they have been shown to be sufficiently equivalent. Optionally, a cross-check design can be employed where Steps 1-6 alternate with Steps 7-14 several times to provide repeated measurements and increased accuracy. Optionally, in a cross-over design can increase measurement accuracy, Steps 7-14 can be employed at location 1 and Steps 1-6 conducted at location 2, and then Steps 7-14 go to location 2 and Steps 1-6 go to location 1. Many other procedural variations and test designs are possible and will be understood by those skilled in the art as being within the scope of the invention.

In the process described, the counters and timers can be located wherever it is convenient. Both counters and timers can be located at a remote computer and connected to the sales environment by any convenient method including, but not limited to, hard wires, telephone links, infrared links and radio or microwave links. In addition, the calculations described above can be performed manually by reading the counters and timers or they can be performed on a computer using a variety of programs.

A Method for Coordinating Elements of a Multimedia Sales Enhancing Campaign

Advertisers who employ multimedia sales campaigns are vulnerable to system failures if the elements of the campaign are not adequately coordinated. Scent is a powerful memory jogger, but if an inappropriate scent is combined with the wrong picture, substantial damage to a brand might be expected and the advertising campaign may be ineffective.

In a multimedia sales enhancing campaign employing scent, it is necessary to coordinate the scent delivery systems with the other multimedia elements such as light boxes, TV players, audio players and the like even if the elements are not co-located or physically near one another.

One solution to the coordination problem is to employ radio frequency identification (RFID) tags. An RFID tag contains an antenna and a small radio transmitter with broadcasts an identification code. A central processor coordinates the tag numbers, and the scent delivery device is activated only when the tags in a multimedia campaign match entries in a database or when tags contain the same identification code.

An iButton™ or similar device may be used in the present invention. The iButton™ is a computer chip enclosed in 16 mm stainless steel can and includes an interface, allowing up-to-date information to travel with a person or object. The steel button can be mounted virtually anywhere because it is rugged enough to withstand harsh environments, indoors or outdoors. It may be attached to a key ring, watch, or other personal items and used daily for applications such as access control to buildings and computers. Because iButton™ tags contain a computer chip, they can therefore carry more information than an RFID tag. iButton™ tags are currently applied to tag animals and in access control, asset management and the like. An iButton™ tag can be attached to each member of a multimedia sales enhancing campaign. The scent delivery could be programmed to be activated only upon detecting a match in the contents of the tags.

A barcode on each element of a multimedia sales enhancing campaign along with an appropriate barcode reader could act to coordinate the elements of the campaign. All elements of a campaign would need to match sufficiently to enable the scent delivery system to be turned on.

Similarly, a chip can be designed to connect with other chips via wires or wireless links where each chip is attached to one element of the multimedia sales enhancing campaign. A master chip on the scent machine or external coordination device checks for an appropriate match among all the elements of the multimedia campaign. Where an appropriate match is found, the scent delivery system is enabled.

A Method of Enhancing the Sale of a Product During a Shopping Experience

The shopping experience today is increasingly hectic. Advertisers must use all available means to reach a shopper and influence a buying decision. Traditional methods such as wall signs and fancy displays are growing ever more ineffective as shoppers rush past the familiar media. Yet, many shoppers use a cart or a carrier container to collect goods prior to purchase and transport them to a checkout register or area where the products can be purchased. Carts or containers are commonly found in grocery stores, drug stores, convenience stores, gas stations, vending areas and the like. The products in the cart or carrier container uniquely define the current interests of the customer.

If the product in a shoppers cart or their wallet such as a frequent shopper card or carrier are electronically tagged with, for example, barcodes, RFID or iButton™ tags, an electronic profile of the customer's current interests can be constructed. For example, a shopping cart full of cosmetics defines an interest in beauty products, but a shopping cart full of doorknobs, doorbells and screwdrivers defines an interest home repair.

A decision support system accesses the profile built from the tagged products in the shopping cart or carrier in order to suggest other products that may be of interest to the shopper. For example, the shopper with cosmetics may also be interested in diet pills while the shopper with the cart full of doorknobs may be interested in a door mat as well.

As the shopper travels through a sales environment, he or she may encounter strategically placed advertising zones containing scenting zones within the advertising zones. In the advertising zone, the contents of the shopping container may be scanned. Based on a profile from the decision support system, a multimedia advertising system displays a picture, hologram, sound or other medium stimulating one or more of the senses of touch, hearing, sight or sound corresponding to the suggested product and a scent delivery system delivers a pre-selected scent or scent delivery program.

The scent delivery system can be chosen from among several commercial scent delivery systems such as those supplied by ScentAir™. Alternatively, a specialized scent delivery system can be constructed to evaporate or vaporize a scent and deliver the scent into a scenting zone. In a most preferred embodiment, the scent delivery device is capable of delivering multiple scents for pre-selected, variable times and to pre-selected concentrations to a scenting zone, a specific, contained zone within a sales environment.

The efficiency of a multimedia advertising system which reads the contents of a shopping container or shopper card or credit card can be assessed using methods similar to those described in previous embodiments. A baseline first sales rate is determined when the scent delivery devices are turned off and a second sales rate determined when the scent delivery device is turned on. The difference between the two sales rates provides an estimate of a sales lift due to scent according to equations 5 or 6, as appropriate.

A Method for Enhancing Reaction to a Product Through a Communication Device Using Scent

As traditional advertising declines in effectiveness, advertisers must seek other ways to communicate with potential customers. Today, many or most people use electronic communication devices including telephones or cell phones, computers connected to the Internet, personal digital assistants and the like. These communication media naturally employ a multimedia message stimulating primarily the senses of sight and sound. The addition of a scent delivery appliance to a communication device can add a whole new dimension to the medium and provide advertisers with a unique method to communicate their value to a customer.

Exploiting communication devices as multimedia advertising platforms involves selecting a product, communicating with a site containing product information, delivering an appropriate scent or scent program and providing a means to purchase or react to the product through the communication device.

In one embodiment of the present invention, product information is contained on a site reachable by a communications device. The site contains information in text, picture or sound formats about the product and provides an option to request scent information about the product. One example of a site is a website on the Internet which can be reached via a computer also connected to the Internet. Another example is a cell phone or a personal digital assistant or the like which can wirelessly connect with a site on the Internet.

Scent information is delivered by an appliance connected to the communication device. Upon a request for scent information being received from the communication device by the site, a signal enabling scent delivery is sent by the site to the scent delivery appliance. Scent is then delivered from pre-loaded cartridges or cassettes, for example, into a scenting zone located near the scent delivery appliance.

The scent delivery appliance can be chosen from among several commercial scent delivery systems such as those supplied by ScentAir™. Alternatively, a specialized scent delivery appliance can be constructed to evaporate or vaporize a scent and deliver the scent into a scenting zone using appropriate fans, ducts and louvers. In a most preferred embodiment, the scent delivery appliance is capable of delivering multiple scents for pre-selected, variable times and to pre-selected concentrations to a scenting zone.

Through the site, the communication device also provides a means to purchase the product. Alternatively, the communications device provides a means for gathering reaction to a product. For example, journalists can be given access to a selected site and can experience a product through multimedia involving at least text, pictures, sound and scent. Then, the same journalists can react to the product experience by issuing press releases or writing stories about the product. Reaction may be measured subjectively or objectively, for example by measuring the number of hits on a website.

A Method for Measuring the Effectiveness of Enhancing Reaction to a Product Through a Communication Device Using Scent

Measuring the effectiveness of a multimedia sales enhancing campaign with the addition of scent and which uses a communication device, involves initially collecting sales information without scent and then collecting sales information with the scent component enabled. Comparison of the two sales rates gives the lift in sales due to scent.

The effect of scent on sales effectiveness is measured by collecting information about the number of visits that occur to various portions of the product information site and correlating that information with product sales. To enable the effectiveness measurement, the site collects the following information during a measurement time-slice (TS1) from various users who connect to the site via the communication device:
1. the number of visitors (V1) to the product information site,
2. the number of visitors (V2) who elect to receive scent information,
3. the number of products (P1) purchased by visitors who did not receive scent information, and
4. the number of products (P2) purchased by visitors who received scent information;
   Using the information collected a first reaction frequency can be calculated according the following equation for visitors who did not request scent information:
   FRF=P1/V1/TS1 Eq. 7.
   For potential customers who received scent information, a second reaction frequency can be calculated according to the following equation:
   SRF=P2/V2/TS1 Eq. 8.
   The difference in the sales rates is the lift in sales frequency (LSF) due to the addition of scent to a multimedia advertising method employing a communications device. The sales lift can be calculated according to the following equation:
   LSF=SRF−FRF Eq 9.
   LSF multiplied by a time duration is an estimate of the sales increment due to adding scent to the multimedia advertising campaign, and can be directly monetized for a merchant or other advertising customer.
   A Multimedia Product Promotion Method Enhanced by Scent

Product promotion involves putting information into the hands of potential consumers in a form that stimulates their interest in the product and motivates them sufficiently to remember the product and to want to purchase the product when it is encountered. Existing promotional systems such as signs, TV spots and the like are growing less effective as they grow more numerous and more common. As a result, a product promotional activity must seek new outlets and new media forms for continued success. Incorporation of scent in to multimedia product promotion offers an attractive option. Scent is a powerful memory enhancer and in combination with other media provides an unusually effective method for product promotion.

A product promotional system involves incorporating scent into communication media that have been used primarily for entertainment in the past. Communication media suitable for product promotion according to the method include digital media, film media, TV, radio, telephones, records, audio tapes, VHS cassettes, DVD's, a computer or other electronic devices.

The product promotional method incorporates the following elements into a package containing the communication medium:
1. reference to the product to be promoted;
2. one or more media which stimulate(s) one or more of the senses of taste, touch, hearing or sight, and
3. a scent delivery system that stimulates the sense of smell with a scent or a scenting program selected to represent the product or promotional idea.

One non-limiting example of a promotional product according to this invention is a promotional DVD for a sausage product. The DVD contains pictures and sounds of the sausage product before, after and during cooking and includes pictures and audio instructions on cooking methods and temperatures to achieve the optimum product, stimulating the senses of sight and sound. A booklet provides similar text information, stimulating the sense of sight. Incorporation of scent corresponding to the various stages of cooking provides a powerful new experience, leading to different and presumably more memorable experience.

Another non-limiting example would be the incorporation of scent into a record or audio tape which contains references to a product or an emotion. One non-limiting example of a product promotional record or audio tape is a tape or record with a song which contains one or more references to a product. If the record or audio tape is enhanced by scent or a scent program that is delivered in conjunction with the product reference, a new experience can be created, enhancing the potential customer's memory or knowledge of the product.

Yet another non-limiting example is a computer mouse rigged to vibrate when it receives a signal from a website containing product information. The same or similar signal that simulates vibration triggers delivery of a scent or scent program simultaneously. The scent delivery device can be optionally incorporated into the mouse or optionally a stand-alone device connected to the computer.

Scent delivery devices that are suitable for the present application can be chosen from among several commercial scent delivery systems such as those supplied by ScentAir™. Alternatively, a specialized scent delivery appliance can be constructed to evaporate or vaporize a scent and deliver the scent into a scenting zone using appropriate fans, ducts and louvers. In a most preferred embodiment, the scent delivery appliance is capable of delivering multiple scents for pre-selected, variable times and to pre-selected concentrations to a scenting zone.

A scent delivery device can be a stand-alone unit connected to the multimedia device or combined with the multimedia device. One non-limiting example is a scent delivery box that is connected by a cable to a DVD player. The DVD player is optionally modified to communicate with the scent delivery box and deliver a scent or scent program when the scent delivery box receives a signal from the DVD. Another non-limiting example is a telephone that delivers a scent when a product related telephone number is dialed. The scent delivery box can optionally be a stand-alone device or optionally incorporated into the telephone.

A Brand Identity Enhancing Method Involving Scent Delivery

Scent is a powerful memory stimulator, but for scent to be used successfully in advertising, a specific scent or scent program needs to be associated with a product in a consumer's memory. The process for creating the association between a product and a scent involves creating an environment in which a potential customer can experience a product.

A brand identity enhancing method involving scent may contain the following elements:
1. A brand name or brand identity;
2. A location where a group of people gather;
3. An airborne scent or scent program that is associated with the brand name or brand identity; and
4. A scent delivery system that delivers the brand associated scent or scent program in conjunction with non-scent advertising media or sinage, or audio or graphics.
   A non-limiting example of a company with a brand name or brand identity might be ScentAir™ or ScentAndrea™ or Proctor & Gamble with Tide™, a laundry product or Crest™, a toothpaste or Hershey's™ milk chocolate.

Non-limiting examples of suitable locations where people gather include weddings, fund raisers, corporate meetings, trades shows, large retail spaces, a feature film with a product and its related scent and the like.

Non-limiting examples of multimedia advertising can include a feature film with a product and its related scent, television or radio spots, print ads, lighted displays, gobos, projected images, audio spots or recorded sounds, product models and the like.

Delivery of a scent or a scent program at a social gathering, wedding, fund raiser, corporate meeting, trade show, large retail space and the like in conjunction with other multimedia advertising provides an opportunity for a potential consumer to associate a scent with a product, an advertising campaign or a brand.

Optionally, the scent delivered by the scent delivery device is a single scent or multiple scents. With multiple scents, a scent delivery program is possible where delivery involves multiple scents delivered for pre-selected, variable times and to pre-selected concentrations to a scenting zone.

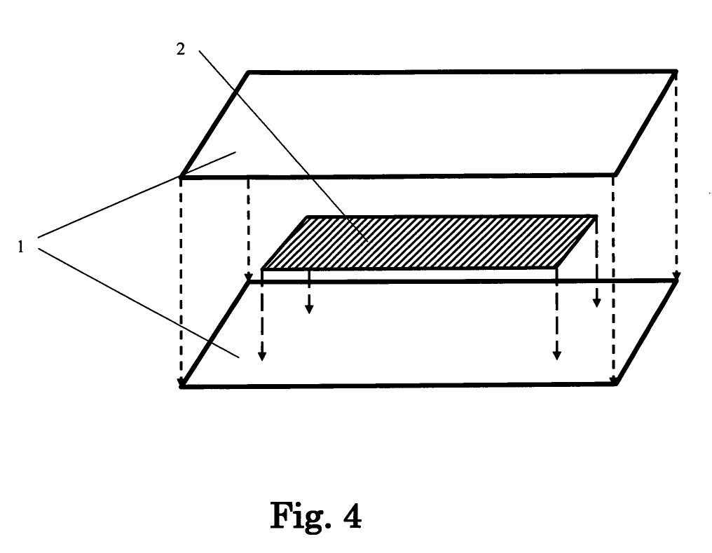

In developing a brand association, scent delivery can be by any convenient device. A scent delivery device can be a stand-alone unit. Optionally, the delivery device can be connected to other multimedia devices or combined with an appropriate multimedia device. Optionally, the delivery device can be a strip, as depicted in FIG. 4 with an overcoat, or barrier film 1 on either side of a scent delivery strip 2. Further, scent delivery devices can be chosen from among several commercial scent delivery systems such as those supplied by ScentAir™. Alternatively, a specialized scent delivery appliance can be constructed to evaporate or vaporize a scent and deliver the scent into a scenting zone using appropriate fans, ducts and louvers. In a most preferred embodiment, the scent delivery appliance is capable of delivering multiple scents for pre-selected, variable times and to pre-selected concentrations to a scenting zone.

A Personal Scent Delivery Device

In addition to providing a lift to sales in a multimedia sales experience, scent can provide a person with a personal brand. The personal brand can provide a unique identity so that other people recognize a person when they enter or leave a room for example. Additionally, a personal brand can provide a personal experience or response such as inducing a restful or aggressive attitude depending on the desires of an individual or the current situation.

A personal brand or a personal experience can be provided by delivering scent into a scenting zone around a person. A personal scent delivery system contains the following elements:
1. a scenting zone located around a person or individual;
2. a container holding at least one, two, or more scents;
3. a control device for selecting a scent for delivery
4. a delivery device which delivers the selected scent or scent program into the region around the person
5. a control device that delivers a new scent
6. There can be a single scent device for each fragrance and it can be chosen like perfume based on the desired effect one wants to achieve:
   a. at timed intervals
   b. according to a preset program
   c. on command of a person.

A scenting zone around a person is chosen to be large enough to provide a scent experience to a person, but small enough to avoid scenting an entire room or offending another person with an alternative scenting desire.

Optionally, the scent delivered by the scent delivery device is a single scent or multiple scents. With multiple scents, a scent delivery program is possible where delivery involves multiple scents delivered for pre-selected or variable times and to pre-selected concentrations to a scenting zone.

A scent is chosen to provide the desired experience. A personal brand scent may be a specific scent such as a unique perfume or a combination of scents. To induce a calming environment, jasmine or chamomile can be delivered in a scent program designed to avoid personal scent insensitivity. To induce weight loss, rosemary or eucalyptus scents can be delivered in a scent program designed reduce appetite or increase appetite. Other examples will be apparent to those skilled in the art.

Scent delivery can be by any convenient device. A scent delivery device can be a stand-alone. Optionally, the scent delivery unit is connected to other multimedia devices or combined with an appropriate multimedia device. A scent delivery device can take any appropriate format. Non-limiting examples of formats include pendants, CD players, a mouse, telephones, decorative objects and traditional formats such as a VCR.

Further, scent delivery devices can be chosen from among several commercial scent delivery systems such as those supplied by ScentAir™. Alternatively, a specialized scent delivery appliance can be constructed to evaporate or vaporize a scent and deliver the scent into a scenting zone using appropriate fans, ducts, dampers and louvers or air convection systems driven by heat, including body heat. In a most preferred embodiment, the scent delivery appliance is capable of delivering multiple scents for pre-selected, variable times and to pre-selected concentrations to a scenting zone.

Scents can be loaded into the scent delivery device by a person. Individual scents are provided as separate cartridges that fit into the scent delivery device, enabling the person to customize the scent program at will.

A control device provides the person with control of the scent delivery times or scent delivery program, allowing the person to choose the scent or scent program. Optionally, the scent program can be altered or personalized by selecting appropriate values on the delivery device.

A Strip-Like Scent Delivery Device

Advertisers are challenged to find means to attract a customer's attention. Multimedia advertising or sales promotion employing scent is an effective means to cause customers in a shopping environment to notice a product or brand. One solution is to provide scent as part of a product display. A time-saver for a store owner or distributor would be to incorporate the scent delivery program for a product into the shipping packages for a product.

A scent delivery device configured as a strip, sheet or similar device with substantial surface area and which can be attached to a product or package would solve the problem. Such a product may be made of:
a. a polymeric material configured with an extended surface area;
b. a scent compatible with the polymeric material;
   i. which can be incorporated into the polymeric material;
   ii. is characteristic of a selected product or brand;
   iii. which releases the scent at a controlled rate;
c. a removable covering device which is impermeable to scent and which permits scent delivery only upon removal.
   The polymeric material can be shaped into any selected form or shape by any convenient method including molding, coating or other suitable processes. Polymeric material includes but is not limited to polyethylene, polypropylene, polycarbonate, cellulose or cellulose derivatives and the like. Polymeric material can also be preformed material such as paper or other porous or microporous structures.

Scent can be incorporated into the polymer by any convenient method including impregnation, precipitation, co-precipitation, coating or other suitable process. Optionally, one surface of the device can be coated with a suitable adhesive, allowing the completed device to stick to a package, product surface, shelving, or display.

A barrier layer over the device substantially prevents scent release until the barrier is removed. Barrier layers and barrier packages are known in the packaging and coating art and include, but are not limited to, metal foil film, coated metal foil films, multilayer barrier films such as those used in food packaging and the like.

One embodiment of a strip-like scent delivery device comprises a strip-like scent delivery media which contains scent or scents for a product. The strip-like scent delivery media is initially sandwiched between two barrier films to form a closed scent delivery package where the barrier films serve to substantially prevent release of scent outside the package. The closed scent delivery package is delivered to a commercial zone in the same shipping carton as the product corresponding to the scent or scents. Optionally, the scent delivery package can be adhesively coupled to the shipping carton. When the shipping package is opened and prepared for display, the barrier film is broken or opened releasing the enclosed scent or scents. A key advantage of a strip-like scent delivery package is that coordination of scent delivery and other advertising media is easily and reproducibly achieved.

Measuring the Advertising or Promtional Efficiency of a Strip-Like Scent Delivery Devcie

The advertising or promotional efficiency of the scent strip and be assess as follows. For the discussion that follows, a first sales rate is measured with the non-scent related multi-media sales enhancing campaign in place and active, but the scent delivery is a placebo strip which does not contain scent. The second sales rate is equivalent but the scent delivery device is turned on.
1. Both the first and second counters are set to zero at the beginning a first measurement time-slice (TS1).
2. When a potential customer enters the scenting zone, the customer detector identifies appropriate behavior. Appropriate behavior is predefined and includes, but is not limited to, changes in eye, hand or body movements, changes in heart rate, changes in the electrical or electromagnetic spectrum of the potential customer or changes in the physical position of a potential customer.
3. A first counter is triggered and the counter counts all potential customers according to preset criteria with in the time-slice, forming a value FCV1.
4. A second counter is located at a check-out or other location where a product is purchased. The second counter is triggered when the selected product is purchased and counts accumulate during the time-slice forming a value, SCV1.
5. If time-slice for the first sale rate, baseline measurement and the second sale rate measurements are constant, the first sales rate, FSR, can be calculated as follows:
   FSR1=SCV1/FCV1 Eq. 1
6. If the time-slices for the first sales rate measurement and second sales rate measurement are not equal, the first sale rate measurement can be calculated as
   FSR2=SCV1/FCV1/TS1 Eq. 2
7. When baseline measurements are completed, the advertising campaign is augmented with scent by replacing with placebo scent delivery strip with an active device which delivers scent. As the next phase of the sales enhancing campaign is initiated and both counters are set to zero in order to initiate the second sales rate measurement which also initiates a second time-slice measurement, TS2.
8. When a potential customer enters the scenting zone containing the advertising campaign, the customer detector identifies appropriate behavior and a scent or scent program is delivered. Appropriate behavior is predefined but is the same behavior measured in Step 2.
9. A first counter is triggered and the counter counts all potential customers according to appropriate preset criteria with in the time-slice, forming a value FCV2.
10. A second counter is located at a check-out or other location where a product is purchased. The second counter is triggered when the selected product is purchased and counts accumulate during the time-slice forming a value, SCV2
11. If time-slice for the first sale rate, baseline measurement and the second sale rate measurements are constant, the second sales rate, SSR, can be calculated as follows:
   SSR1=SCV2/FCV2 Eq. 3
12. If the time-slices for the first sales rate measurement and second sales rate (SSR) measurement are not equal, the second sale rate measurement can be calculated as follows:
   SSR2=SCV2/FCV2/TS2 Eq. 4
13. The increment in sales (ISR) due to scent augmentation of the multimedia advertising method can be estimated by comparing the first sales rate with the second sales rate. If the time-slices, TS1 and TS2, are the same, the following equation defines the sales increment.
   ISR1=SSR1−FSR1 Eq. 5.
14. If the time-slices, TS1 and TS2, are unequal, the following equation defines the sales increment.
   ISR2=SSR2−FSR2 Eq. 6.
   ISR2 multiplied by a time duration is an estimate of the sales increment due to adding scent to the multimedia advertising campaign, and can be directly monetized for a merchant or other advertising customer.

Several variations of the theme are possible. Steps 1-6 and Steps 7-14 can be applied sequentially in a single sales environment. Optionally, Steps 1-6 and Steps 7-14 can be conducted in separate sales environments if they have been shown to be sufficiently equivalent. Optionally, a cross-check design can be employed where Steps 1-6 alternate with Steps 7-14 several times to provide repeated measurements and increased accuracy. Optionally, in a cross-over design can increase measurement accuracy, Steps 7-14 can be employed a location 1 and Steps 1-6 conducted at location 2, and then Steps 7-14 go to location 2 and Steps 1-6 go to location 1. Many other procedural variations and test designs are possible and will be understood by those skilled in the art as being within the scope of the invention.

In the process described, the counters and timers can be located wherever it is convenient. Both counters and timers can be located on a remote computer and connected to the sales environment by any convenient method including, but not limited to, hard wires, telephone links, infrared links and radio or microwave links. In addition, the calculations described above can be performed manually by reading the counters and timers or they can be performed on a computer using a variety of programs.

A Scent Delviery Fan

A scent delivery device may be a fairly complex system. A scent delivery device typically includes (1) a scent reservoir, (2) a scent delivery system to move the scent out of the reservoir in a controlled fashion, (3) a vaporization facilitation component, (4) an air moving device and (5) appropriate air delivery ductwork to deliver the scent to a scenting zone or other desired location. A simplification of a scent delivery device occurs when elements of the device can be integrated. Such integration simplifies on-site or customer conducted system maintenance.

In one embodiment, the fan of the scent delivery system is surrounded by a porous shroud where the shroud contains scent. Examples of shrouds include fan grills and screens. Scent can be incorporated into the shroud by impregnation or coating, or insertion of a cartridge. Alternatively, the porous shroud can be in contact with a fluid in a scent reservoir, whereby the shroud draws the scent from the reservoir and delivers the scent into an air stream of an air moving device. Additionally, a heater can be incorporated into or around the shroud to encourage vaporization of the scent.

A substantial simplification of a scent delivery device can be achieved by integrating into a single device: (1) a scent reservoir, (2) a scent delivery system to move the scent out of the reservoir in a controlled fashion, (3) a vaporization motivating component, (4) an air moving device. One embodiment achieving the goal of scent device simplification is to integrate the scent reservoir, a scent wicking function, a scent vaporization function and the fan. An advantage of using the fan as the integrated component is that maintenance of a scent device is greatly simplified, enabling customers, distributors or store owners to maintain their own devices.

A fan integrating a scent reservoir, scent delivery system, scent evaporation and air movement system may contain the following:
a fan
   which contains a central hub;
   which rotates around an axis;
   with blades made from porous material;
   which encourages air flow transversely through one or more blades in order to delivery scent into the air stream;
   but where the edges which rotate perpendicular to the axis are optionally nonporous.
a reservoir located in the central hub which contains one or more scents;
a connection between the reservoir and the blades which delivers scent or scents to the blade from the reservoir upon rotation.
   An advantage such devices is that they can be expected to deliver scent over an extended period and they can use scents soluble in a variety of solvents so long as the solvent is compatible with the materials forming the blades.

A simpler scent delivery system for integrating a scent reservoir, scent delivery system, scent evaporation and air movement systems contains the following:
a fan
   which rotates around an axis;
   with blades made from polymeric material;
a scent
   which corresponds to a product or brand and
   which can be incorporated into the polymeric material and
   which is released at a controlled rate.
      An advantage of such a device is that it does not contain seals or other devices that may fail.

Scent can be incorporated by impregnation, precipitation, co-precipitation, coating or other similar process that are known in the art.

The polymeric material is chosen for convenience and can include but is not limited to polymers such as polyethylene, polypropylene, polycarbonate, polymer combinations and the like. Choosing the appropriate polymer can enable an appropriate fan manufacturing method such as molding as one non-limiting example.

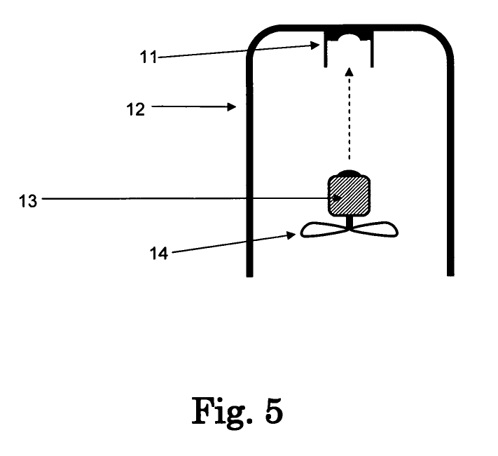

In one embodiment of the simpler scent delivery system, illustrated in FIG. 5, a fan 14 which is loaded with scent or scents is mounted on a motor 13. By turning on a switch, a customer or consumer can activate scent delivery Scent or scents can be chosen to correspond to products. Scent or scents can also be chosen to be pleasing to an individual. A variety of formats for a simpler scent delivery system can be envisioned and include both formats in which the fan blade is enclosed, and formats similar to a pinwheel, where the fan blade is not enclosed.

In another embodiment of an integrated scenting device, a fan 14 is mounted on a motor 13 and the motor is mounted on a contact 11 similar to those found on common light bulbs. In this format, the integrated scenting device can be mounted in a standard light socket. An especially preferred embodiment includes a recessed light fixture 12 which hides or contains the integrated scenting device. The device can be turned on or off using a room light switch, remote or similar device. With the switch turned on, the motor is energized, the fan turns and scent is delivered from the fan. A motion detector can be used to turn on the fan when focused on the intended zone.

A Scent Delviery Apparatus for Converting a Fan to a Scent Delivery System

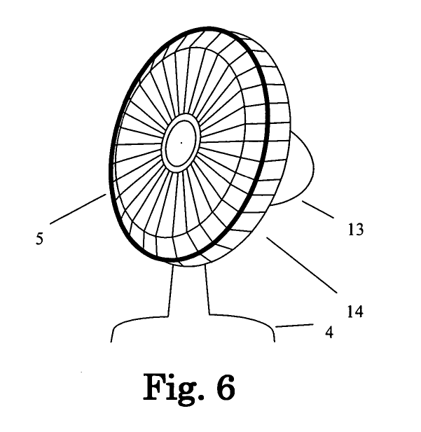

A simplified version of a scent delivery fan contemplated by the present invention may be made by placing a scent receptacle 6 on the protective cage 5 of a fan. While in the present invention, the receptable may be placed at any effective location on the protective cage, it is preferred that it be placed at a central point such as the hub of the protective cage. An example of a fan intended to be used in the present invention is depicted in FIG. 6. A typical fan 14 has a base 4, although a base is not necessary, a motor 13, and a protective cage 5 around the fan blades. Virtually any fan may be used according to the present invention, as long as it has a protective cage.

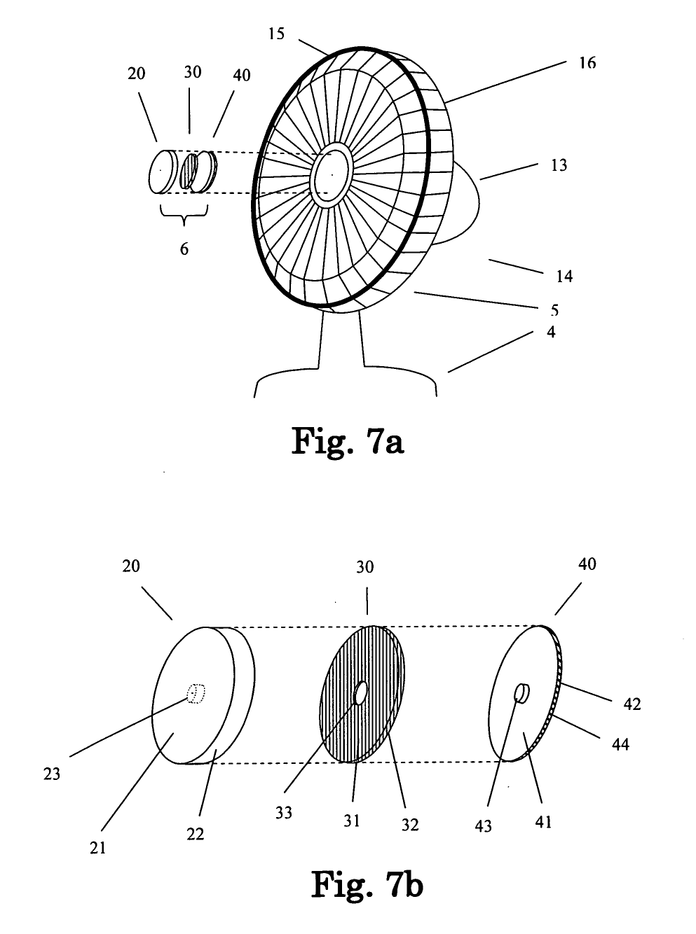

The protective cage 5 may be constructed of plastic, metal, or any suitable material, and serves to provide a support on which to place the scent receptacle 6, as demonstrated in FIG. 7a. In one embodiment of the present invention, the protective cage 5 has a front section 15 and a back section, 16. In embodiments of the invention where the scent receptacle is placed inside the protective cage, it is preferable that the front section be easily opened, for example by means of locking hinges. Variations of fan structure and design are readily known to those of skill in the art.

FIG. 7b shows the main contents of the scent receptacle 6, which are the receptacle cover 20, the scent cartridge 30, and the base 40.

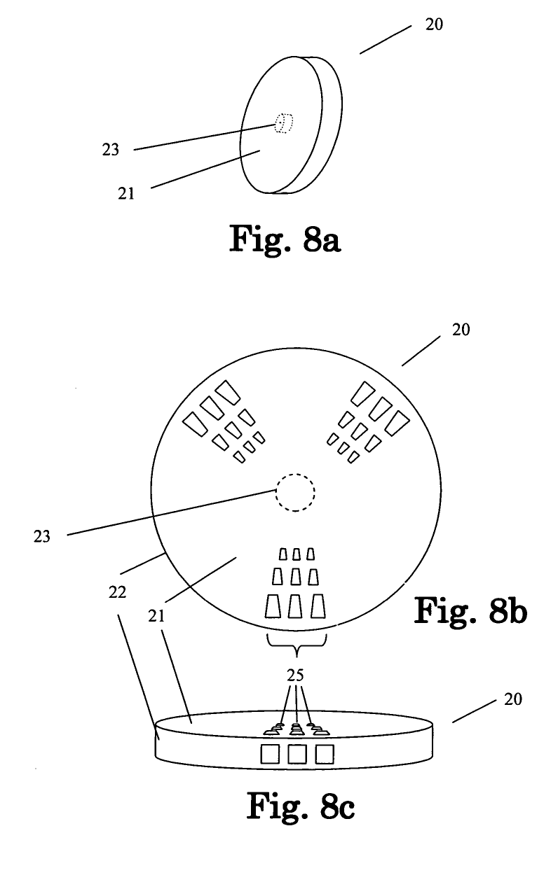

The receptacle cover 20, as shown in further detail in FIG. 8a, is shaped to accommodate the scent cartridge 30 and has an outer surface 21 and a side surface 22. As illustrated in FIGS. 8b and 8c, one or more apertures 25 are positioned on either the outer surface, the side surface, or both surfaces. The apertures serve to allow scent to pass from the scent cartridge inside the receptacle to the environment outside the receptacle. Optionally, the cover may have a means for adjusting the apertures to allow more or less scent to pass through the receptacle cover. For example, the apertures could be adjusted by means of one or more plates that may be slid over all or a portion of the apertures. The cover may also have a structure for attaching to the base, for example a centrally located threaded shaft 23 or threads located on the circumference of the cover.

The scent cartridge 30 is designed to fit inside the receptacle cover and be mounted on the base. The scent cartridge may be constructed from any number of suitable materials known in the art, so long as a scent is provided that may be effectively dispersed from the scent receptacle. The two components required for the scent cartridge are a scent and a medium for supporting for the scent. Although it is technically possible for a scent to be self-supporting, such substances are rare. Therefore, a support medium may be used to hold the scent and to allow the scent to be dispersed through the apertures.

One example of a medium contemplated by the present invention is a wick that provides for transport of the scent. Examples of wicks include cardboard and nylon wicks. In a preferred embodiment of the invention, a wick is in contact with an evaporative surface. In another embodiment of the invention, the scent is dispersed in a volatile solvent at the evaporative surface.

Another example of a medium contemplated by the present invention is a composition containing the scent. Such a composition may be either solid or fluid, continuous or non-continuous, aqueous or hydrocarbon based. For example, a composition according to the present invention could be an emulsion including effective amounts of one or more of a dispersing agent, a thickening agent, a surfactant, and a solvent. In a preferred embodiment of the present invention, the composition may take the form of a continuous phase in which the scent is dispersed. The medium for supporting the scent could also be a composition such as an aqueous or hydrocarbon based solution to which scent may be added. If aqueous, suitable organic solvents and dispersing agents may be used to assist in dispersion of the scent, including alcohols, ketones, halogenated solvents, and esters. If the composition is hydrocarbon based, preferably the composition is essentially volatile, so that the composition will readily evaporate. Where the composition is not sufficiently solid, the scent cartridge may incorporate a container to hold the composition, and a means for releasing the composition from the container, such as a valve or distributor.

Examples of suitable hydrocarbon based solvents and dispersing agents include aliphatic, aromatic, and terpene hydrocarbons.

Examples of thickening agents include: (1) starches, gums, casein, gelatin, and phycocolloids, (2) cellulose derivatives, (3) polyvinyl alcohol and carboxy-vinylates, and (4) bentonite, silicates, and colloidal silica.

Examples of surfactants include nonionic surfactants, anionic surfactants, amphoteric surfactants, and cationic surfactants. If desired, a surfactant should be selected to provide a stable composition for the intended life of the scent cartridge. The type and effective quantity of the surfactants will depend on the solubility of the components in the composition.

In a preferred embodiment of the present invention, the scent is added to a solid composition such as a polymer. The scent may either be dispersed within the polymer composition itself or the polymer can be designed to be impregnated by the scent. A particularly preferred embodiment of the present invention employs a composition of low density polyethylene plastic containing ethyl vinyl acetate. Ethyl vinyl acetate may also be used without polyethylene. Scent may be added to the polymer composition and molded in a desired shape. Another embodiment of the present invention utilizes polymer beads or other shapes to form a packed bed.

Other substances that may be used as the composition for the scent medium in the scent cartridge include polymer foams and gels.

Foamed polymers offer the possibility of minimizing the amount of polymer while simultaneously creating void spaces in which the scent may be placed. Open or closed-cell foams may be used according to the present invention depending on the volatility of the scent or solvent in which the scent is dispersed. Examples include foamed plastics such as foamed polyethylene and foamed polyurethane.

A preferred embodiment of the present invention employs an aqueous gel as the support for the scent. For example, a gel medium could be made by combining a gelling agent such as a gum or polysaccharide, a cross-linking agent such as sodium or potassium citrate or a halide, water, a surfactant, and a solvent such as glycol. The scent may be mixed into the gel thereby forming the scent cartridge of the present invention.

As would be apparent to one having familiarity in the art of scent compositions, any number of additives may be added to the scent medium composition. These additives could include colors, surfactants, preservatives, desiccants, and spreading agents and thickeners.

The present invention is not intended to be limited by a particular scent. However, several specific scents have demonstrated superior performance. The present invention is therefore especially intended to be used with scents of sandalwood, eucalyptus, citronella, and tea. Frequently, the scent is provided as an oil, but may also be an ester or an extract in alcohol. In a preferred embodiment of the invention, a scent composition contains an effective amount of a scent oil from sandalwood, eucalyptus, citronella, or tea.

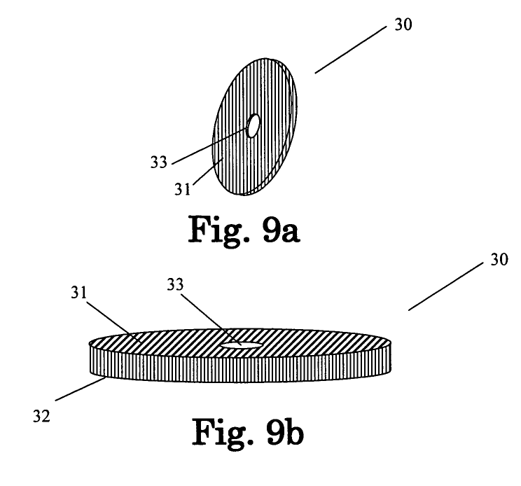

The scent cartridge 30 may be replaceable or rechargeable in the present invention. The scent cartridge is positioned in between the cover 20 and the base 30. The scent cartridge may he held in place by means of a shaft extending from the base, through the scent cartridge, and secured by the cover. An example of such a scent cartridge according to the present invention is depicted in FIGS. 9a and 9b. The scent cartridge has a surface 31 positioned adjacent the cover and a surface 32 positioned at the base. These surfaces may be the same or different. Optionally, the scent cartridge has an opening 33 that may be used to accomplish a friction mounting of the cartridge on the shaft on the receptacle base. The cartridge may have ridges or other structures to increase the surface area of the cartridge for optimal scent transport.

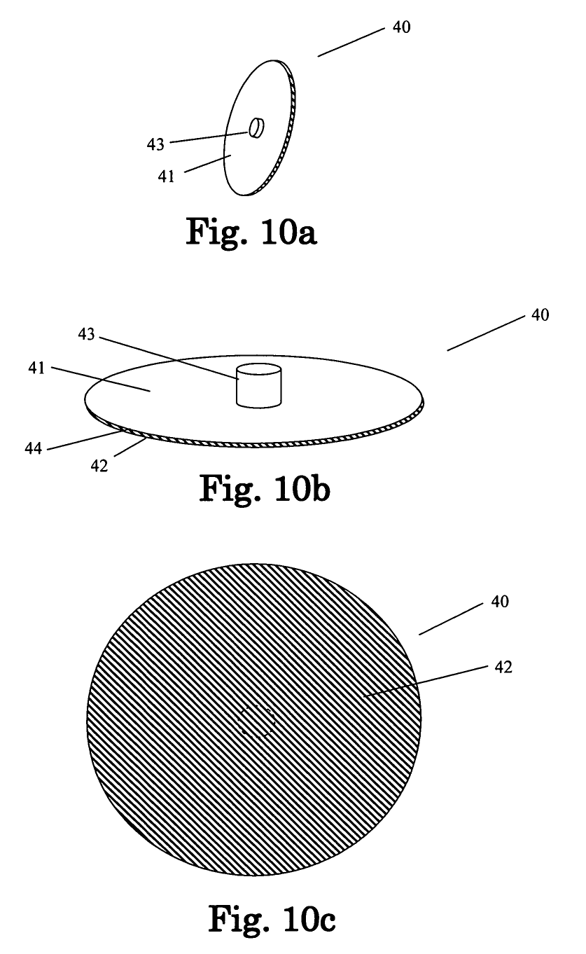

FIGS. 10a-10c show the base 40 of the scent receptacle. The base has a means for attaching the base to the fan cage 5, such as an adhesive 42, located on the outer surface 44 of the base. Means for attaching the base of the receptacle to a fan cage could be any effective means apparent to one of skill in the art, including, for example, adhesives, screws, and nuts and bolts. In the case of screws, a mounting may be attached to the base allowing for screws to secure the base to the fan cage.

The inner surface 41 of the base is intended to be adjacent to the scent cartridge. The outer surface 44 is intended to be positioned near or on the fan cage. The base may also have a means for securing the cover 20, such as a shaft 43 extending from the center of the base that may be connected to the cover. Optionally, this shaft may connect with an adjoining threaded shaft 23 extending from the center of the cover. Another example of means for securing the cover in the present invention is threads enabling the cover to be screwed directly onto the base. The threads could be located on a shaft or on the circumference of the base.

In a preferred embodiment of the invention, the receptacle cover is removable from the base so that the scent cartridge may be replaced or recharged. In this embodiment of the invention, the means for securing the base to the cover should be selected to allow the receptacle to be repeatedly opened and closed. However, the present invention also includes embodiments where the cover is not intended to be opened once the scent cartridge has been placed inside.

Optionally, an LED wand may be mounted inside the protective cage, wherein the LED wand may be programmed to display a message. The LED wand rotates, and the message can be changed by remote-control. This embodiment is particularly useful in analysis or enhancement of sales in multi-sensory advertising.

### EXAMPLE 1

A scent-delivery device is provided having:
(a) a scent cartridge containing a scent and a medium for supporting the scent;
(b) a receptacle holding the scent cartridge, the receptacle having a base and a cover, where the cover is optionally capable of being opened to replace the scent cartridge;
(c) a fan having a protective cage, the protective cage having an inside surface and an outside surface; and
(d) means for attaching the receptacle to a surface of the protective cage.
   The scent cartridge of Example 1 contains a scent selected from the group consisting of citronella, eucalyptus, and sandalwood. The scent is mixed into a medium for supporting the scent, comprising low density ethyl vinyl acetate, a surfactant, and an organic solvent. The medium is molded into a wafer shape with ridges for added surface area, and has a hole in the middle and designed to be placed onto the shaft inside the scent receptacle.

The cover of the scent receptacle is plastic having a circular surface and lateral edge on the circumference normal to the circular surface. The circular surface has an array of apertures extending radially from the center of the cover with the size of the apertures increasing towards the circumference of the cover. Apertures are also located in the lateral edge of the cover, allowing for additional circulation. The base of the receptacle is made of plastic and has a centrally located tapered shaft extending upwards on which the scent cartridge is frictionally mounted. Inside the cover is a threaded opening to which the receptacle base is screwed. The scent cartridge is placed over the shaft and held in place by friction fitting around the shaft so that air can circulate on both sides of the scent cartridge. After mounting the scent cartridge on the base of the receptacle, the cover is screwed shut. The base is then attached to the front, outer surface of the protective cage of the fan by a set of screws.

Scent is dispersed upon operating the fan. When the scent is exhausted, the cover of the receptacle can be opened and the scent cartridge replaced.

A rotating LED wand is mounted on the rotor of the fan. The LED is programmed to display a message when the blades are rotating. It is characteristic of the present invention to have the LED display the product associated with the scent dispersed.

### EXAMPLE 2

A scent-delivery device is provided having:
(a) a scent cartridge containing a scent and a medium for supporting the scent;
(b) a receptacle holding the scent cartridge, the receptacle having a base and a cover, where the cover is optionally capable of being opened to replace the scent cartridge;
(c) a fan having a protective cage, the protective cage having an inside surface and an outside surface; and
(d) means for attaching the receptacle to a surface of the protective cage.
   This example is similar to the previous example with the following distinctions:
(1) The medium for supporting the scent is a polymer resin or gel and the scent is citronella.
(2) The scent receptacle is placed inside the protective fan cage by opening the fan cage. An adhesive is used to attach the base of the receptacle to the hub inside the fan cage immediately in front of the fan.
(3) The fan cage has a system of hinged locks that allows for the cage to be opened for access to the scent receptacle.

The foregoing description of the invention is intended for purposes of example. Many other variations in procedure and design are possible and are apparent by those skilled in the art as being within the scope of the invention.

### PATENT REFERENCES

1. U.S. Pat. No. 6,103,201 Green; Dennis E. “Propeller air freshener”.
2. US20020130146A1 Borut, Severine et al. “Automatic air freshener with dynamically variable dispensing interval”
3. U.S. Pat. No. 6,664,254 Rogozinski; Wallace. “Odor-eliminating composition”.
4. EP0495631A3 Bernaducci, Ernest et al. “Air freshener device with visual signaling means”
5. U.S. Pat. No. 5,220,741 Burgeson; John R. “Temperature regulated scent dispenser”
6. U.S. Pat. No. 4,788,787 “Scent propagation device”
7. U.S. Pat. No. 5,303,496 “Scent distributing method for hunters”
8. U.S. Pat. No. 5,963,302 “Process and device for diffusing perfumes that accurately correspond to events or scenes during cinematographic representations and the like”
9. U.S. Pat. No. 6,239,857 “Process and device for the synchronous addition of odors to visual and/or acoustic stimulation”
10. U.S. Pat. No. 6,152,829 Jaidka; Sandeep “Device and process for producing effects”
11. US2001/0048641A1 Kaslon, David A. “Scent emitting device”
12. US2004/0065748 Sada, David P. “Scent Strip”
13. U.S. Pat. No. 4,629,604 Spector; Donald “Multi-aroma cartridge player”
14. U.S. Pat. No. 5,398,070 Lee; Dong H. “Smell emission control apparatus for television receiver”
15. EP 0295129 B1 Machida, Hirojl “Method for supplying aromas, apparatus there fore and facilities provided with same.”
16. WO9801175A1 SCHWARTZ, Gary et al. “AROMA THERAPY DIFFUSER”
17. US2002/0124409A1 “Aroma therapy delivery system”
18. Vending Times. 43(11): pp-pp, 2003.
19. U.S. Pat. No. 6,026,987 Burnett; Sean C. et al. “Aroma dispensing system”
20. U.S. Pat. No. 5,771,778 MacLean, IV; John A. “Marketing system”
21. U.S. Pat. No. 3,844,057 Johnson, Frank “Advertsing and Sales Promotion Method and Apparatus”
22. U.S. Pat. No. 6,669,092 Leanheart, Steven J. “Display Apparatus”
23. U.S. Pat. No. 5,069,876 Oshinsky, C. “Combined Scent and Audio Point of Sale Display Unit”
24. US2003/0091466A1 Benko, K. “Method and apparatus for releasing fragrance”
25. US2004/0103028A1 Method And Systems Of Advertising
26. US2003/0066073A1 Methods & Systems Of Interactive Advertising.
27. WO03061716A1 Triplett, C. et al. Method And Apparatus For Positioning A Wick Material In A Vapor-Dispensing Device
28. U.S. Pat. No. 4,419,326 Santini; Thomas F. Vapor Dispersing Process
29. WO0230220A1 Porous Wick For Liquid Vaporizers
30. U.S. Pat. No. 5,857,620 Liquid Dispenser With Integral Wick/Emanator Assembly
31. U.S. Pat. No. 5,749,520 Air Freshener Dispenser Device With Capillary Wicking Means
32. WO04014440A1 Reece, C. et al. An Apparatus To Better Distribute An Insect Repellent Or Fragrance

### OTHER REFERENCES

33. “Mass Media: Killing The Goose That Lays Golden Eggs”. BusinessWeek Online (Readers Report). Aug. 2, 2004.

What is claimed is:

1. A scent-delivery apparatus for converting a fan to a scent dispenser, the scent-delivery apparatus comprising:
(a) a scent cartridge containing a scent and a medium for supporting the scent;
(b) a receptacle for holding the scent cartridge, the receptacle having a base and a cover, wherein the cover is optionally capable of being opened to replace the scent cartridge; and
(c) means for attaching the base of the receptacle to a fan having a protective cage whereby the receptacle is attached to the protective cage.

2. The scent-delivery apparatus of claim 1 wherein the receptacle has one or more apertures through which scent may pass.

3. The scent-delivery apparatus of claim 1 wherein the cover of the receptacle has one or more apertures through which scent may pass.

4. The scent-delivery apparatus of claim 2 wherein the apertures may be adjusted to vary the amount of scent passing through the apertures.

5. The scent-delivery apparatus of claim 1 wherein the medium for supporting the scent is one or more selected from the group consisting of solid plastic, foamed plastic, a packed bed, a gel, an emulsion, a solution, and a wick optionally used in combination with a solvent.

6. The scent-delivery apparatus of claim 1 wherein the medium for supporting the scent is one or more selected from the group consisting of low-density polyethylene, ethyl vinyl acetate, and poly vinyl chloride.

7. The scent-delivery apparatus of claim 1 containing one or more scents selected from the group consisting of citronella, eucalyptus, and sandalwood.

8. The scent-delivery apparatus of claim 1, wherein the means for attaching the base of the receptacle to the protective cage is selected from the group consisting of: adhesive located on the base of the receptacle and screws.

9. The scent-delivery apparatus of claim 1, wherein:
(a) the base of the receptacle has an outer surface positioned near the protective cage and an inner surface positioned near the scent cartridge, the inner surface having a centrally located shaft extending from the inner surface;
(b) the scent cartridge has an opening allowing the centrally located shaft to pass through the scent cartridge; and
(c) the scent cartridge is held in place by a friction fitting around the shaft.

11. A scent-delivery device comprising:
(a) a scent cartridge containing a scent and a medium for supporting the scent;
(b) a receptacle holding the scent cartridge, the receptacle having a base and a cover, where the cover is optionally capable of being opened to replace the scent cartridge;
(c) a fan having a protective cage, the protective cage having an inside surface and an outside surface; and
(d) means for attaching the receptacle to a surface of the protective cage.

12. The scent-delivery device of claim 11, wherein the protective cage comprises opening means providing access to the inside surface of the protective cage.

13. The scent-delivery device of claim 11, wherein the protective cage comprises a front portion and a rear portion, means for attaching the front portion to the rear portion, and one or more hinges allowing the front portion to be swung open from the rear portion.

14. The scent-delivery device of claim 11, further comprising an LED wand mounted inside the protective cage, wherein the LED wand may be programmed to display a message.

15. The scent-delivery device of claim 14, wherein the LED wand rotates and the message can be changed by remote-control.

16. The scent-delivery device of claim 11, wherein the protective cage has an axis of radial symmetry and the receptacle is attached at the axis of the protective cage.

17. The scent-delivery device of claim 11, wherein the cover of the receptacle is circular in shape and has an array of apertures extending radially from the center of the cover with the size of the apertures decreasing towards the center of the cover.

18. A method for measuring sales enhancement of a product, the method comprising:
a. selecting a product;
b. providing a sales enhancing campaign for the product, the sales enhancing campaign incorporating
(1) a scent cartridge containing one or more scents and a medium for supporting scent;
(2) a receptacle holding the scent cartridge, the receptacle having a base and a cover, where the cover is optionally capable of being opened to replace the scent cartridge;
(3) a fan having a protective cage, the protective cage having an inside surface and an outside surface; and
(4) means for attaching the receptacle to a surface of the protective cage;
c. providing a commercial zone in which a product is displayed for sale;
d. providing an advertising zone that is optionally within the commercial zone, but may be overlapping or separate;
e. providing one or more detectors configured to detect the presence of a potential customer in the commercial zone;
f. calculating a first sales rate determination in which the advertising zone does not contain the sales enhancing campaign;
g. calculating a second sales rate determination in which the advertising zone does contain the sales enhancing campaign;
h. comparing the first sales rate with the second sales rate, from which one may determine the effect of the sales enhancing campaign.

19. The method according to claim 18 where the first sales rate determination comprises:
a first measurement time-slice;
a first counter that counts a number of potential customers in the commercial zone selected from the group of potential customers entering, leaving, and present in the commercial zone during the first measurement time-slice;
a second counter that counts the number of sales of the selected product within the selected measurement time-slice; and
a first sales rate (fsr) computed by dividing the value of the second counter (scv1) divided by the value of the first counter (fcv1) divided again by the value of the first measurement time-slice (ts1), expressed mathematically as fsr=scv1/fcv1/ts1.

20. A method for dispersing a scent comprising:
(a) placing a scent cartridge containing one or more scents and a medium for supporting scent into a scent receptacle having a base and a cover;
(b) closing the cover of the scent receptacle over the base thereby enclosing the scent cartridge;
(c) mounting the base of the scent receptacle onto the cage of a fan;
(d) operating the fan and disseminating the content of the scent cartridge.
